(19) United States
(12) Patent Application Publication
(10) Pub. No.: US 2008/0193478 A1
(43) Pub. Date: Aug. 14, 2008

(54) Inactivated Poliomyelitis Vaccine Derived From Sabin Strain Of Polio Virus

(76) Inventors: Rajesh Jain, New Delhi (IN); Anil Chawla, New Delhi (IN); Ganesh Kumraj, New Delhi (IN)

Correspondence Address: LADAS & PARRY LLP, 26 WEST 61ST STREET, NEW YORK, NY, 10023, US

(21) Appl. No.: 11/661,523

(22) PCT Filed: Aug. 27, 2004
(86) PCT No.: PCT/IN2004/000267

§ 371 Date: Nov. 20, 2007

(51) Int. Cl. A61K 39/13; A61K 39/295; A61P 31/12

(52) U.S. Cl. 424/202.1; 424/217.1

(57) ABSTRACT

An inactivated Polio Vaccine derived from Sabin strain for safe and effective immunization against Poliomyelitis is provided. A process of preparation for such vaccine and formulations thereof are also provided. Administration of the vaccine of the present invention along with other antigens provides immunization not only against polio infection but also against other pathogens causing Hepatitis C. Hepatitis D. Hepatitis E. Meningitis A. Meningitis B. Meningitis C. Meningitis W. Meningitis Y. Pnemococcal (23 valent or more). Smallpox, Typhoid, Bacille Calmette Guerin, Tuberculosis. Human Immunodeficiency Virus. Anthrax or the like, to which children or adults not immunized earlier are susceptible, particularly to which children are susceptible.

### FIELD OF THE INVENTION

The present invention relates to a Polio Vaccine, preferably in inactivated form, derived from Sabin strain for safe and effective immunization against Poliomyelitis, a process for the preparation of such vaccine, and formulation thereof.

The Polio Vaccine formulations of the present invention can be used along with one or more other antigens to provide immunization not only against polio infection but also against other pathogens such as those causing Hepatitis C, Hepatitis D, Hepatitis E, Meningitis A, Meningitis B, Meningitis C, Meningitis W, Meningitis Y, Pnemococcal (23 valent or more). Smallpox, Typhoid, Bacille Calmette Guerin, Tuberculosis, Human Immunodeficiency Virus, Anthrax, or the like, to which children or adults when not immunized earlier are susceptible, particularly to which children are susceptible.

The Polio Vaccine formulation prepared according to the present invention is very beneficial for primary immunization of children because it not only prevents polio infection but also other types of infection to which children or adults when not immunized earlier are susceptible, particularly to which children are susceptible.

### BACKGROUND OF THE INVENTION

Poliomyelitis (also commonly referred to as ‘Polio’) is an acute infection that involves the gastrointestinal tract and, occasionally, the central nervous system. It is acquired by fecal-oral transmission. In the prevaccine era, infection with poliovirus was common, with epidemics occurring in the summer and fall in temperate areas. The incidence of poliomyelitis declined rapidly after the licensure of inactivated polio vaccine in 1955 and oral polio vaccine in the 1960s. The last cases of indigenously acquired polio in the United States occurred in 1979. Although a polio eradication program led to elimination of polio in the Western Hemisphere, where the last case associated with wild poliovirus was detected in 1991, outbreaks of vaccine-derived poliovirus type 1 occurred in the Dominican Republic and Haiti in July 2000 and in the Philippines in 2001. In spite of these recent outbreaks, the global polio eradication initiative has reduced the number of reported polio cases worldwide by >99% since the mid-1980° s, and worldwide eradication of the disease appears feasible in the near future. Clinical manifestations of poliovirus infection range from asymptomatic (the majority of injections) to symptomatic, including acute flaccid paralysis of a single limb to quadriplegia, respiratory failure, and, rarely, death.

Polio is caused by three types of quite stable viruses namely Type 1, Type 2, and Type 3 belonging to the family of enteroviruses. There are different polio vaccines available in the market. These vaccines are trivalent containing a mixture of all the three types of poliovirus so as to confer immunity against all of them.

One type of Polio Vaccine available is Inactivated Polio Vaccine (IPV) based on Salk strain (Salk IPV). The vaccine contains all the three types of polio virus, inactivated by formalin. The major advantage of Salk IPV is that it can be incorporated with other childhood immunizations like DPT. However the Salk IPV has the several disadvantages such as the requirement of stringent standards for growing the Salk strain of poliovirus, and the potential risk posed due to accidental release of live virulent polio viruses into the environment while handling of virulent strain of polio viruses during its production process. Moreover, vaccine formulations comprising the Salk strain of poliovirus are costly, and have a comparatively lesser potency.

Another type of Polio Vaccine available is the Oral Polio Vaccine (OPV) based on Sabin strain. This orally administered Polio vaccine consists of live inactivated strains of polio virus. Such vaccine compositions comprising of live inactivated Sabin strains of polio virus are comparatively cheaper than Inactivated Polio Vaccine (IPV) based on Salk strain.

The OPV is commonly used because of the ease of availability of monovalent bulks, larger number of manufacturing facilities, ease of administration, and most significantly, the manufacture of OPV does not pose any risk of accidental release of virulent polio viruses into the environment. However some of the disadvantages associated with the OPV are the perishability of OPV at about temperatures above minus 20° C., the reversal of attenuated strains to virulent strains in vaccine composition, and vaccine associated paralytic poliomyelitis.

U.S. Pat. No. 5,639,649 by Almond et al relates to the construction of vaccines against rhinoviruses and enteroviruses, particularly polioviruses, by the introduction of defined mutations into their genomes. These mutations attenuate the virulence of wild type viruses and can further attenuate existing live attenuated vaccine virus strains, thereby making them less likely to revert to virulence.

U.S. Pat. No. 5,618,539 by Dorval et al pertains to stabilized viral vaccines, particularly live viral vaccines for poliomyelitis, comprising an aqueous solution of a live virus and a stabilizing amount of a compound containing at least two amino or imine groups, such as basic amino acids e.g. lysine.

The U.S. Pat. No. 4,525,349 by Montagnon et al describes a process for large-scale production of poliomyelitis vaccine. However, it does not mention the stabilization process of the polio virus prior to the inactivation stage.

Hence, there still exists an unmet need for developing cheaper vaccine compositions comprising live attenuated polio virus, which can be administered by injection and are devoid of toxicity or have reduced toxicity, without compromising the immunogenicity.

Moreover, although different types of polio vaccines are known in the art, there is no prior art disclosure of a stable and efficacious formulation comprising Sabin strain derived inactivated polio vaccine and other antigens effective against pathogens causing infections among children or adults, particularly children, which are devoid of toxicities or are less toxic, and are immunogenic.

### SUMMARY OF THE INVENTION

It is an objective of the present invention to provide an Inactivated Polio Vaccine derived from Sabin strain for effective immunization against Poliomyelitis.

It is also an objective of the present invention to provide a process for the preparation of an Inactivated Polio Vaccine derived from Sabin strain for effective immunization against Poliomyelitis characterized in that the Sabin strain of the Polio virus are first stabilized by adding a stabilizer, and then inactivated by an inactivator.

According to a process of the present invention, the monovalent bulk of polio virus is initially stabilized by adding stabilizer followed by inactivation comprising of the following steps:
i) initial stabilization of bulk of polio virus by adding stabilizer,
ii) addition of inactivator such as formaldehyde (formalin) or beta-propiolactone to each of the filtered and purified monovalent pool or only heating the pool,
iii) incubation at about 37° C. upto 48 hours and then at 2° C. to 8° C. upto 12 days in a single cycle before further processing,
iv) testing for free formaldehyde content or the inactivator concentration performed after every 12 hours during the inactivation process and the desired concentration level is maintained by intermittent readjustments,
v) optionally doing a second filtration after the process of inactivation.

According to another process of the present invention, the monovalent bulk of polio virus is initially stabilized by adding stabilizer followed by inactivation comprising of the following steps:
i) initial stabilization of bulk of polio virus by adding stabilizer,
ii) addition of inactivator such as Formaldehyde (formalin) or beta-propiolactone or only heating,
iii) incubation at 37° C. for 4 hours followed by chilling at 2° C. to 8° C. for 20 hours,
iv) repetition of the incubation and chilling cycle up to 12 times so that total exposure at 37° C. does not exceed 48 hours during 12 days of incubation,
v) testing for free formaldehyde content or the inactivator concentration performed after every 12 hours during the inactivation process and the desired concentration level is maintained by intermittent readjustments,
vi) optionally doing a second filtration done after the process of inactivation.

It is another objective of the present invention to provide a formulation comprising an Inactivated Polio Vaccine derived from Sabin strain for immunization against Poliomyelitis characterized in that the strains of the Polio virus are first stabilized by adding a stabilizer, and then inactivated by an inactivator.

It is a further objective of the present invention to provide a stable and effective Polio Vaccine formulations with other antigens which upon administration provides immunization not only against polio infection but also against other pathogens causing Hepatitis C. Hepatitis D, Hepatitis E, Meningitis A, Meningitis B, Meningitis C. Meningitis W. Meningitis Y, Pnemococcal (23 valent or more), Smallpox, Typhoid, Bacille Calmette Guerin, Tuberculosis, Human Immunodeficiency Virus. Anthrax, or the like, to which children or adults are susceptible, particularly to which children are susceptible.

### DETAILED DESCRIPTION OF THE OF THE INVENTION

The present invention provides an Inactivated Polio Vaccine derived from Sabin strain for effective immunization against Poliomyelitis.

The present invention provides a Polio Vaccine derived from Sabin strain for effective immunization against Poliomyelitis prepared from Sabin seed strain or Sabin strain monovalent bulk suspensions used for blending of trivalent oral polio vaccine or by growing Sabin strain Polio viruses (from monovalent bulk used for trivalent OPV) in vero cell line or any suitable cell culture characterized in that the Polio virus is first stabilized by adding a stabilizer, as herein described, and then inactivated by adding formaldehyde (Formalin) according to a specific embodiment of the invention.

The Polio vaccine of the present invention eliminates the disadvantages, associated with known Polio vaccines, OPV and Salk IPV.

In an embodiment, the Sabin strain derived inactivated polio vaccine is trivalent containing a mixture of all the three types of polio virus namely type 1, Type 2 and Type 3.

In another embodiment, the Sabin strain derived inactivated polio vaccine is bivalent containing a mixture of any two types of polio virus selected from Type 1, Type 2 and Type 3.

In an essential embodiment of the present invention, the polio viruses are stabilized before inactivating the viruses to retain their shape, since the three-dimensional shape of the viruses are highly important for their antigenicity (immunogenicity). The stabilization process before inactivation protects the already weak Sabin strains from getting deformed or damaged during inactivation process.

The stabilization process of the monovalent bulk of poliovirus involves an addition or stabilizer(s) selected from but not limited to the group comprising of sucrose, trehalose, arginine hydrochloride, gelatin, magnesium chloride, aluminium chloride, and disodium EDTA, used either alone or in combination thereof, to retain the antigenicity of the vaccine during inactivation process.

In an embodiment, the inactivation process of each of filtered and purified monovalent pool involves addition of inactivator such as but not limited to formaldehyde (formalin) or beta-propiolactone, or mixture thereof preferably at about 0.001% to 0.1%, more preferably at 0.025% concentration, and/or heating and incubation at about 37° C. for optimized number of days.

The present invention provides a process for the preparation of Inactivated Polio Vaccine derived from Sabin strain for effective immunization against Poliomyelitis

In an embodiment, the present invention provides a process for preparation of polio vaccine from Sabin seed strain or Sabin strain monovalent bulk suspensions used for blending of trivalent oral polio vaccine or by growing Sabin strain polio viruses in vero cell line or any suitable cell culture for the preparation of oral polio vaccine characterized in that the polio vaccine prepared is stabilized by a stabilizer and then inactivated by an inactivator.

In a preferred embodiment, the Polio vaccine prepared from Sabin strain, as stated above, may be stabilized and inactivated, according to present invention, by one of the following two processes.

### Process-I:

Stabilization process: The monovalent bulk of poliovirus is initially stabilized by adding stabilizer like Sucrose, Trehalose Fir Arginine hydrochloride, to retain the antigenicity of the vaccine during inactivation process.

Inactivation process: The inactivation process is initiated immediately. Inactivation of each or the filtered and purified monovalent pool is carried out by adding formaldehyde (formalin) at 0.025% concentration or beta-propiolactone and incubation at about 37° C. upto 48 hours and then at 2° C. to 8° C. up to 12 days in a single cycle before further processing. Either of the treatment could be used for inactivation. The test for free formaldehyde content is performed after every 12 hours during the inactivation process and the desired concentration level is maintained by intermittent readjustments. A second filtration is done after the process or inactivation. Consistent inactivation of the virus is monitored and verified.

### Process-II:

Stabilization process: The monovalent bulk vaccines are stabilized using the same procedure as described for Process-I.

Inactivation Process Inactivation is carried out by adding formaldehyde (formalin) at 0.025% concentration or beta-propiolactone and incubation at 37° C. for 4 hrs followed by chilling at 2° C. to 8° C. for 20 hrs. This incubation & chilling cycle can be repeated up to 12 times so that total exposure at 37° C. does not exceed 48 hrs during 12 days of incubation. The test for free formaldehyde content is to be performed after every 12 hrs during the inactivation process and the desired concentration level is maintained by intermittent readjustments. A second filtration is to be done after the process of inactivation. Consistent inactivation of the virus is monitored and verified.

The maintenance of potency/antigenic integrity of highly heat labile Sabin viruses is ensured. The inactivation processes are standardized in such a way that live inactivated virus of each type is completely inactivated and the process ensures intact antigenic structure of Sabin strain. Inactivated vaccine is able to impart adequate immunity in vaccines when compared with standard vaccine. The Sabin strain poliovirus, in present invention, is stabilized before inactivation using stabilizer like sucrose, trehalose, arginine hydrochloride, magnesium chloride, aluminium chloride, and disodium EDTA to avoid degradation of nativeness and antigenic structure. The inactivated Polio Vaccine, prepared according to the present invention, in monovalent, bivalent or trivalent form has required amount of equivalent D-antigen or antigen form which could be compared with an established reference standard of inactivated polio vaccine and/or provide sero-conversion and required protection.

The Sabin strain derived Inactivated Polio Vaccine (SIPV) prepared according to the present invention, possesses the advantages of both OPV and Salk IPV. The process of manufacture does not require growing of virulent strains of polio virus and hence the chances of accidental release of live virulent polio viruses into the environment is very less. Also the strains of Polio virus retain their shape even in the formulations which prevents a compromise on the immunogenicity, since the three-dimensional shape of the viruses are highly important for their effectiveness. Moreover, the SIPV are devoid of disadvantages like vaccine associated paralytic poliomyelitis. Also, the SIPV of the present invention can be effectively combined with other antigens thus providing effective immunization against several diseases at the same time.

The Sabin strain derived Polio vaccine used in the formulations according to the present invention may be trivalent containing a mixture of all the three types namely Type 1, Type 2 and Type 3 of Polio virus or may be a mixture of any two of the said Types.

The administration of the Polio Vaccine formulations prepared according to the present invention would overcome or at least mitigate the problems associated with multiple injections of the different antigens. The formulations provided by the present invention are stable and highly immunogenic which are suitable for administration to children or adults, particularly suitable for administration to children.

In an embodiment of the present invention, the Polio vaccine obtained by the standardized methods is used to provide a multivalent vaccine formulation comprising Sabin strain derived inactivated Polio vaccine (SIPV).

In another embodiment, the Polio vaccine obtained by the standardized methods is used to provide a multivalent vaccine formulation comprising Sabin strain derived inactivated Polio vaccine (SIPV) and a number ‘n’ of other antigens, optionally in combination with an adjuvant comprising one or more aluminum salts, in which the value of ‘n’ is 1 or greater than 1. Preferably the value of ‘n’ is about 1 to 30. More preferably the value of ‘n’ is 2, 3, 4, 5 or 6. The term ‘multivalent’ used herein means vaccine formulation comprising at least two or more antigens.

In yet another embodiment of the present invention, a formulation is provided, wherein the Sabin strain derived inactivated polio vaccine is trivalent containing a mixture of all the three types of polio virus namely Type 1, Type 2 and Type 3, or bivalent containing a mixture of any two types of polio virus selected from Type 1. Type 2 and Type 3.

In yet another embodiment of the present invention, a formulation is provided wherein the vaccine is multivalent.

The other antigens present in the Polio Vaccine formulations according to the present invention are selected from but not limited to those which provide immunity against one or more of the pathogens causing infections like Hepatitis C, Hepatitis D, Hepatitis E, Meningitis A, Meningitis B, Meningitis C, Meningitis W, Meningitis Y, Pnemococcal (23 valent or more), Smallpox, Typhoid, Tuberculosis, Human Immunodeficiency Virus, Anthrax or the like, to which children or adults are susceptible, particularly to which children are susceptible.

The adjuvant(s) used in the present invention include but not limited to aluminium hydroxide, aluminium phosphate, calcium phosphate, oil emulsions such as Freund's emulsified oil adjuvants (complete and incomplete), Arlacel A, Mineral oil, Emulsified peanut oil, adjuvant (adjuvant 65), products from bacteria (their synthetic derivatives as well as liposomes) or gram-negative bacteria, endotoxins, cholesterol, fatty acids, aliphatic amines, paraffinic and vegetable oils, Algammulin, and QS-21. Preferably, aluminium hydroxide and aluminium phosphate is used as adjuvant either alone or in combination thereof.

In an embodiment, the formulations of the present invention additionally comprises of but not limited to preservatives or tissue fixatives or both.

The preservatives used in the formulations of the present invention are selected from but not limited to ethyl mercury, thimerosal, merthiolate, and 2-phenoxy ethanol, used either alone or in combination thereof.

The tissue fixative used in the formulations of the present invention is but not limited to formaldehyde.

In another aspect the invention provides a vaccine formulation comprising Sabin strain derived inactivated Polio vaccine (SIPV) adsorbed to Aluminum Phosphate (AP) and an antigen adsorbed to AP or to Aluminium Hydroxide (AH) or any other suitable adjuvant selected from an antigen providing immunity against one or more of the following pathogens namely Hepatitis C, Hepatitis D, Hepatitis E. Meningitis A. Meningitis B, Meningitis C. Meningitis W, Meningitis Y, Pnemococcal (23 valent or more). Smallpox, Typhoid, Bacille Calmette Guerin, Tuberculosis, Human Immunodeficiency Virus, Anthrax or the like.

In a further aspect of the invention, there is provided a stable and effective vaccine formulation directed to the prevention of at least two diseases, comprising SIPV and at least one other antigens selected from Hepatitis C, Hepatitis D. Hepatitis E, Meningitis A. Meningitis B, Meningitis C, Meningitis W, Meningitis Y, Pnemococcal (23 valent or more), Smallpox, Typhoid, Bacille Calmette Guerin, Tuberculosis, Human Immunodeficiency Virus, Anthrax or the like.

In another embodiment of the present invention, the formulation also includes SIPV compatible antigens like antigens against Meningitis B, Meningitis A and C, or otitis media.

In a further embodiment, the inactivated monovalent viruses are mixed with but not limited to stabilizer or/and preservative to make monovalent, bivalent, trivalent or multivalent inactivated vaccine. The final vaccine may be spray dried & resuspended in ready-to-inject liquids like perfluorocarbons, requiring no refrigeration for storage or preparation before injection.

The formulations prepared according to the present invention may be administered through but not limited to the routes such as oral, transdermal, parenteral, nasal, mucosal, and the like.

In a preferred embodiment of the present invention, the formulation is administered as an injection through the parenteral route, particularly through subcutaneous or intramuscular route.

The example provided below illustrates the embodiments of the present invention. However, it does not intend to limit the scope or the present invention.

### EXAMPLE-1

Seed strain of the same type and passage as used for poliomyelitis vaccine (oral): Oral polio Vaccine (OPV) is propagated to produce monovalent suspension for inactivation.

### Cell Culture:

i) Different cell substrates for propagation of virus include Human diploid cell line, Monkey kidney cells, Vero cell line or any other suitable cell culture.
ii) Large scale production of cells:
1. Preparation of the Manufacturer's working cell bank (MWCB): MWCB in case of primary cell cultures (cells derived from normal tissue and stored frozen at minus 70° C.) or from any other permitted continuous cell line are prepared from the cells & the cells pooled after serial subculture within the specified number of cell cultures.
2. Any of the following systems can be used which ever is suitable, for stepwise increase in the volume of cells.
Roller bottles: Growth in roller bottles differs from that in stationary cultures in distribution of cells on glass surface and maximum attainable cell density. Roller bottle cultures do not deteriorate less rapidly as they can tolerate a density nearly two times higher than that of stationary cultures. Mammalian cells grown in monolayer culture will adapt best to roller bottle technique.
Cell factories: These are stack of culture trays that share a common inlet and outlet port. These provide cells with a growing surface as large as inside of roller bottle, but take up less space inside incubator. These are ideal for adherent cell cultures, have low contamination, risk and are compact.
Roux flasks
Micro-carrier system
   3. Samples for control cell cultures are incubated separately for at least two weeks and these are examined for evidence of cytopathic changes.

### Propagation of Polio Virus in Cell Cultures:

i) Seed lot system:
a) Passage level as followed for Oral polio Vaccine OR
b) Its next passage level as derived for monovalent bulk suspension that could be used to manufacture oral polio vaccine.
1. The vaccine is manufactured on the basis of virus seed lot system. Virus seed lot is a quantity of virus processed together and of uniform composition prepared from seed lot.
2. Sub-culture of the seed virus should not be done more than 10 times counted from a seed lot used for the production of the vaccine on which original laboratory and field tests were done.

### Single Harvest and Monovalent Pools:

The virus is propagated in cell cultures and harvested from cell cultures derived from a single batch of cells and processed together. This is known as single harvest. The single harvests of one type of virus suspension are processed at the same time to get crude form of monova lent pool.

### Filtration or Clarification of Monovalent Pool:

Crude virus suspension of each monovalent pool is purified stepwise through filters of decreasing porosity. The purpose of filtration step is to remove particulate material and other substance that may affect inactivation process as such aggregates tend to increase on standing.

### Concentration of the Monovalent Pool:

Each filtered monovalent pool is concentrated by Ultra-filtration.

### Purification of the Monovalent Pool:

Chromatography with DEAE sepharose/immobilized DNA-ase column/immuno-adsorption column is employed for the purification of the monovalent pool. The purification process reduces consistently the level of cellular DNA from that of initial virus harvest by a factor of at least 10⁸. The D-antigen concentration is determined by ELISA and display of comparable immunogenicity in rats. Accordingly the potency is adjusted. The monovalent pools of Poliovirus are blended to form the final trivalent bulk product.

### Stabilising the Sabin Virus Before Inactivation:

The antigenicity of the vaccine is closely associated with the stability or the native virus antigens during inactivation process. The poliovirus antigenic structure is to the initial stabilized by adding stabilizer like sucrose, trehalose, or arginine hydrochloride.

### Inactivation Using Formaldehyde:

i) Inactivation process is initiated preferably within 24 hours and not later than 72 hours after filtration.
ii) Inactivation of each of filtered and purified monovalent pool is carried out by adding formaldehyde (formalin) at 0.025% and incubation at 37° C. for specific time period as described earlier. The test for free formaldehyde is performed at intervals and the desired concentration level is maintained by intermittent readjustments.
iii) A second filtration is done after the process of inactivation.
iv) Consistent inactivation of the virus is monitored and verified.
v) For testing the extent of inactivation, formaldehyde is neutralized by adding sodium bisulfite and subsequently dialyzing it out.

### Formulation and Manufacturing of the Vaccine:

The purified inactivated bulk is filtered, preferably through 0.22 micron or 0.45-micron filter. The inactivated monovalent viruses is optionally mixed with stabilizer and/or preservative to make monovalent, bivalent, trivalent or multivalent inactivated vaccine.

### Testing the Efficacy of the Vaccine:

The efficacy of the vaccine as manufactured above, as compared with established reference vaccine, is found to be similar in in-vitro and/or in-vivo method.

Various other tests based on the standard accepted methods were carried on the vaccine manufactured above such as pH determination, sterility testing, test for effective inactivation, abnormal toxicity study, volume measurement, in vitro potency determination, endotoxin test, identity test, protein nitrogen content estimation, and determination of stabilizer content and formaldehyde content to insure its effectiveness, safety and potency.

The in vitro potency of the SIPV manufactured above was determined using Sandwich ELISA. Two primary antibodies raised in different species against polio virus were utilized for the same and a second antibody conjugated with HRP is used again at the second primary antibody. The results were analyzed by comparison with reference standard.

The identity test for the vaccine was carried using, direct ELISA. Antigen was coated well in 96 ELISA plates and type specific anti polio sera is employed for determining the presence of all the three types of polio virus in the vaccine.

1-35. (canceled)

36. An inactivated polio vaccine derived from Sabin strain for effective immunization against Poliomyelitis containing at least two types of Type 1, Type 2 and Type 3 of polio virus, prepared from Sabin seed strain, Sabin strain monovalent bulk suspensions or Sabin strain polio said vaccine prepared by a process comprising the steps of: stabilizing the polio virus using a stabilizer and then inactivating by using an inactivator.

37. The inactivated polio vaccine according to claim 36, which is in a form that is suitable for oral, transdermal, parenteral, nasal or mucosal administration.

38. The polio vaccine according to claim 36, wherein the stabilizer is selected from trehalose, arginine hydrochloride, gelatin, aluminium chloride, and disodium EDTA, used either alone or in a combination thereof.

39. The polio vaccine according to claim 36, wherein the inactivator is selected from formaldehyde and beta-propiolactone or a mixture thereof.

40. A process for preparation of a polio vaccine from Sabin seed strain, Sabin strain monovalent bulk suspensions of polio virus or Sabin strain polio viruses comprising the steps of: stabilizing the polio virus using a stabilizer and then inactivating by using an inactivator.

41. A process according to claim 40, wherein the monovalent bulk suspension of polio virus is, initially stabilized followed by inactivation, comprising the steps of:
i) pooling, filtering and purifying cell cultures of monovalent bulk suspensions of polio virus, to obtain pooled polio virus;
ii) stabilizing the pooled polio virus obtained in step i) by adding stabilizer;
iii) adding an inactivator selected from formaldehyde and beta-propiolactone or a mixture thereof;
iv) incubating at about 37° C. up to 48 hours and then at 2° C. to 8° C. up to 12 days in a single cycle before further processing;
v) testing for free formaldehyde content or inactivator content every 12 hours during the inactivation process and maintaining the desired concentration level by intermittent readjustments; and
vi) optionally carrying out a second filtration after the process of inactivation.

42. The process according to claim 40, wherein the monovalent bulk of polio virus is initially stabilized followed by inactivation, comprising the steps of:
i) pooling, filtering and purifying the monovalent bulk suspension of polio virus to obtain pooled polio Virus;
ii) stabilizing the pooled polio virus obtained in step i) by adding stabilizer;
iii) adding an inactivator selected from formaldehyde and beta-propiolactone or a mixture thereof;
iv) incubating at 37° C. for 4 hours followed by chilling at 2° C. to 8° C. for 20 hours;
v) repeating the incubation and chilling cycle up to 12 times so that total exposure at 37° C. does not exceed 48 hours during 12 days of incubation;
vi) testing for free formaldehyde content or inactivator concentration after every 12 hours during the inactivation process and maintaining the desired concentration level by intermittent readjustments; and
vii) optionally carrying out a second filtration after the process of inactivation.

43. The process according to claim 40, wherein the stabilizer is selected from the group comprising trehalose, arginine hydrochloride, gelatin, aluminium chloride, and disodium EDTA, used either alone or in combination thereof.

44. The process according to claim 40, where in the inactivator is formaldehyde or beta-propiolactone or a mixture thereof.

45. The process according to claim 40, further comprising adsorbing the polio vaccine on an adjuvant with 1 to 30 other antigens.

46. The process according to claim 45, wherein the other antigens are selected from a group comprising Hepatitis C, Hepatitis D, Hepatitis E, Meningitis A, Meningitis B, Meningitis C, Meningitis W, Meningitis Y, Pnemococcal (23 valent or more), Smallpox, Typhoid, Bacille Calmette Guerin, Tuberculosis, Human Immunodeficiency Virus, and Anthrax, used alone or in combination thereof.

47. The process according to claim 40, wherein the inactivated polio vaccine is trivalent containing a mixture of Type 1, Type 2 and Type 3 polio virus.

48. The process according to claim 40, wherein the inactivated polio vaccine is bivalent containing a mixture of any two types of polio virus selected from Type 1, Type 2 and Type 3 polio virus.

49. A Sabin strain derived inactivated Polio Vaccine formulation effective for immunization against Poliomyelitis, wherein the vaccine is prepared from Sabin seed strain, Sabin strain monovalent bulk suspensions used for blending of trivalent or bivalent oral polio vaccine or by growing Sabin strain polio viruses wherein the polio vaccine is prepared by a process comprising the steps of stabilizing the polio virus using a stabilizer and then inactivating by using an inactivator, and adsorbing the polio vaccine on an adjuvant optionally with 1 to 30 other antigens.

50. The formulation according to claim 40, wherein the antigens are selected from a group comprising Hepatitis C, Hepatitis D, Hepatitis E, Meningitis A, Meningitis B, Meningitis C, Meningitis W, Meningitis Y, Pnemococcal (23 valent or more), Smallpox. Typhoid, Bacille Calmette Guerin, Tuberculosis, Human Immunodeficiency Virus, and Anthrax, used alone or in combination thereof.

51. A formulation according to claim 49, which comprises one or more of adjuvants selected from a group comprising aluminium hydroxide, aluminium phosphate, calcium phosphate, oil emulsions, Arlacel A, Mineral oil, Emulsified peanut oil adjuvant (adjuvant 65), natural and synthetic products from bacteria, bacterial liposomes, gram-negative bacteria, endotoxins, cholesterol, fatty acids, aliphatic amines, paraffinic and vegetable oils, Algammulin, and QS-21, used either alone or in combination thereof.

52. The formulation according to claim 49, which additionally comprises a preservative, tissue fixative or both.

53. The formulation according to claim 52, wherein the preservative is selected from a group comprising ethyl mercury, thimerosal, merthiolate and 2-phenoxy ethanol, used either alone or in combination thereof.

54. The formulation according to claim 52, wherein the tissue fixative is formaldehyde.

55. A Sabin strain derived inactivated Polio Vaccine formulation for effective immunization against Poliomyelitis, wherein the vaccine is prepared from Sabin seed strain, Sabin strain monovalent bulk suspensions used for blending of trivalent or bivalent oral polio vaccine or by growing Sabin strain polio viruses wherein the polio vaccine is prepared by a process comprising the steps of stabilizing the polio virus using a stabilizer and then inactivating by using an inactivator.
